(19) United States
(12) Patent Application Publication
(10) Pub. No.: US 2019/0343188 A1
(43) Pub. Date: Nov. 14, 2019

(54) INTERACTIVE MALE UNDERWEAR

(71) Applicant: Lukasz KONARSKI, Wroclaw (PL)

(72) Inventor: Lukasz KONARSKI, Wroclaw (PL)

(21) Appl. No.: 16/349,600

(22) PCT Filed: Oct. 31, 2017
(86) PCT No.: PCT/PL2017/050053

§ 371 Date: May 13, 2019

(30) Foreign Application Priority Data
Nov. 14, 2016 (PL) P.419445

(51) Int. Cl. A41B 9/02; A61F 7/00; A41D 13/005

(52) U.S. Cl. CPC A41B 9/02; A61F 7/007; A41D 13/0053; A61F 2007/0048; A61F 2007/0236; A61F 2007/0078; A61F 2007/0086; A61F 2007/0094; A61F 2007/0075

(57) ABSTRACT

The subject of the invention is interactive male underwear, intended to control the temperature of the surface of the skin of the male testicles, wherein reducing the temperature of the surface of the skin of the male testicles contributes to the treatment of male infertility in particular such as oligospermia, aspermia, asthenospermia, azoospermia, nekrospermia, normospermia and teratospermia. The male underwear comprises bearing element (EN) connected with the belt (LK) using connectors (LK), however at least one Peltier cell (8) of the skin temperature control system (UC) is mounted on a bearing element (EN). The bearing element (EN) is made of woven belt narrowed at the bottom in the capital “U” letter shape, however the upper part of the bearing element (EN) is connected directly with the belt (PA), and the D lower part is connected with the belt (PA) using connectors (LK).

### TECHNICAL FIELD

The subject of the invention is interactive male underwear, intended to control the temperature of the surface of the skin of male testicles, wherein reducing the temperature of the skin surface of male testicles contributes to the treatment of male infertility in particular such as oligospermia, aspermia, asthenospermia, azoospermia, nekrospermia, normospermia and teratospermia.

### BACKGROUND ART

Personal cooling device known from the patent application US No. US2012031582, cools the surface of groin directing cooler air to this part of the body. The device is located in the immediate vicinity of user's groin and can be worn on clothes. The device has a housing containing air inlet opening, air outlet opening, inner chamber. The movement of air, inside the inner chamber, is via the air inlet opening, through the inside and outside chambers, through the air outlet opening in the direction of groin area. The device contains an electric fan that forces the air flow. The external surfaces of the housing are smooth and the housing has a “V” shaped tapered part which makes it easier to put it in the user's groin, between the user's legs. The air outlet opening contains many bellows-type slots that provide wide air separation and therefore more effective cooling.

The essence of the underwear according to the invention is that has the bearing element is connected with the belt using connectors, however at least one Peltier cell of the skin temperature control system is mounted on a bearing element. The skin temperature control system is mounted on the bearing element or the skin temperature control system is mounted on the belt and at least one Peltier cell of the skin temperature control system is mounted on the bearing element.

Preferably, the bearing element is made of woven belt narrowed at the bottom in the capital “U” letter shape, however the upper part of the bearing element is connected directly with the belt, and the lower part is connected with the belt using connectors.

Preferably, a hole is made in the centre of the bearing element.

Preferably, the bearing element is made of plastic narrowed at the bottom and with a recess from the top in the shape of capital U letter, however the upper and lower part of the bearing element is connected with the belt, moreover ventilation holes are made in the bearing element.

Preferably, the bearing element is equipped with a pocket, where the skin temperature control system is placed.

Preferably, the bearing element is made of heat conducting material.

Preferably, the belt is made of heat conducting material.

Preferably, the belt is equipped with a heat exchanger fitted with heat sink and fan.

Preferably, internal layer of the belt is made of a thermal insulating material.

Preferably, the heat exchanger is a wide belt.

Preferably, the connector is made of heat conducting material.

Preferably, the belt, connector and bearing element are made of fabric, preferably of cotton fabric, soaked in silver nanoparticles.

Preferably, the skin temperature control system is fitted with a processor connected to the radio module, temperature sensor, moisture sensor, pulse oximeter, GSR sensor, at least one Peltier cell using a key engaging the cell and power supply via the power control system.

Preferably, the power supply is a battery or accumulator connected to the charger.

Preferably, the radio module is connected wirelessly with a mobile application.

Interactive male underwear is used to treat male infertility. It is made of textile material, optimally of cotton with added silver nanoparticles. Such fabric is a very good conductor of heat and moreover, it can be characterized by antybacterial properties. Because of application of the cooling elements together with a temperature sensor, the underwear is able to continuously maintain the reduced temperature male testicles. Scientific studies show that reducing the temperature of male tescticles by 2 degrees leads to significant improvement of male semen. The underwear is comfortable and ergonomic to wear.

### BRIEF DESCRIPTION OF DRAWINGS

Subject of the invention in its embodiment is shown on figure, where

FIG. 1 shows the interactive underwear with bearing element made of woven belt from front,

FIG. 2 shows the underwear with bearing element made of woven belt from top,

FIG. 3 shows the underwear with bearing element made of woven belt from side,

FIG. 4 shows the interactive underwear with a hole in the bearing element made of woven belt from front,

FIG. 5 shows the interactive underwear with bearing element made of plastic from front,

FIG. 6 shows the interactive underwear with bearing element made of plastic from top,

FIG. 7 shows the interactive underwear with bearing element made of plastic from side, and

FIG. 8 shows skin temperature control system.

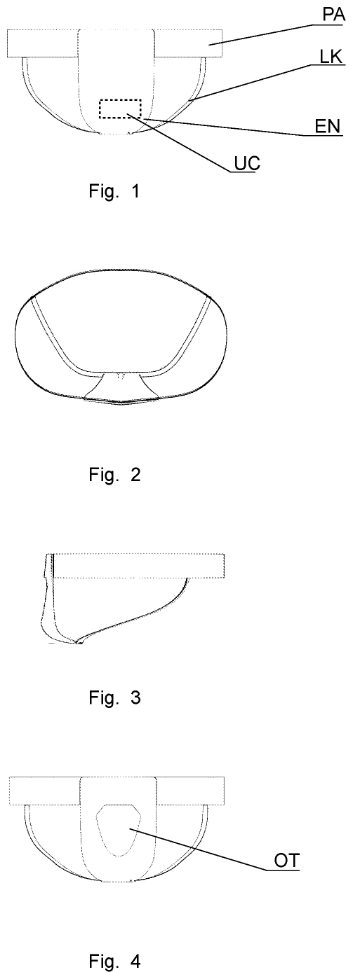

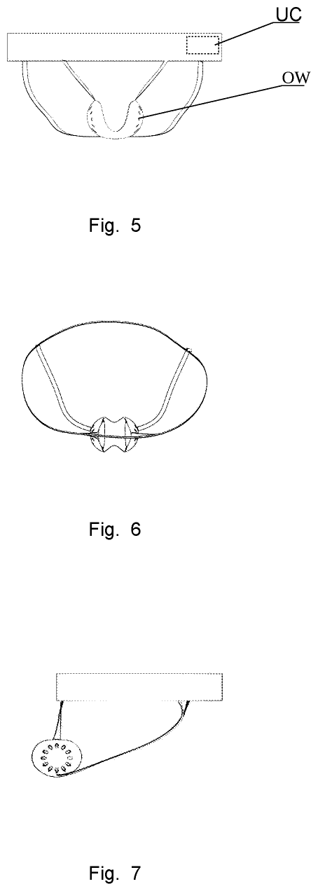

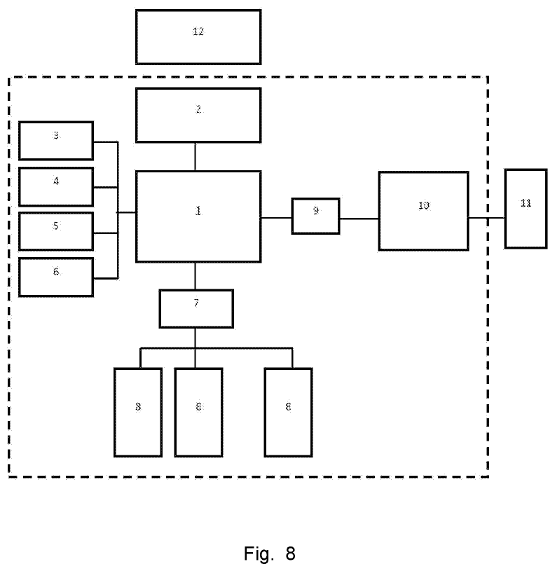

### DESCRIPTION OF EMBODIMENTS

### Example 1

The interactive male underwear has the bearing element EN connected with the belt PA using connectors LK, wherein three Peltier cells 8 of the skin temperature control system UC and the skin temperature control system UC are mounted on the bearing element EN. The bearing element EN is made of woven belt narrowed at the bottom in the capital “U” letter shape, however the upper part of the bearing element EN is connected directly with the belt PA, and the lower part is connected with the belt PA using connectors LK. The bearing element EN, belt PA and connectors LK are made of heat conducting material. In this embodiment, the heat exchanger is provided in a form of a wide belt PA, internal layer of which is made of a thermal insulating material. The skin temperature control system US is fitted with a processor 1 connected to the radio module 2, temperature sensor 3, moisture sensor 4, pulse oximeter 5 to measure saturation and pulse, GSR sensor 6 to measure skin resistance, at least one Peltier cell 8 using a key engaging the cell 7 and power supply 10 via the power control system 9. Power supply 10 is a battery.

### Example 2

Interactive male underwear made as in the first embodiment with the difference that the centre part of the bearing element EN has a hole OT, and the bearing element EN is equipped with a pocket where the skin temperature control system UC is located. The belt PA, connector LK and bearing element EN are made of cotton fabric soaked in silver nanoparticles. In this embodiment, the power supply 10 is an accumulator connected to the charger 11. Moreover, the radio module 2 is connected wirelessly with a mobile application 12.

### Example 3

Interactive male underwear made as mentioned in the first embodiment with the difference that the bearing element EN is made of plastic narrowed at the bottom and with a recess from the top in the shape of capital U letter, however the upper and lower part of the bearing element EN is connected with the belt PA using the connectors LK, moreover a ventilation hole OW is made in the bearing element EN. In addition, the inner side of the bearing element EN is covered with a heat conducting layer applied in the process of metallising. The skin temperature control system UC is mounted on the belt PA, and the Peltier cells 8 of the skin temperature control system UC are mounted on bearing element EN. The belt PA is equipped with a heat exchanger fitted with heat sink and fan.

The temperature sensor 4 located in the underwear measures the temperature of male testicles and sends this information to the microprocessor 1 which controls the current flowing through the Peltier cell 8. The cell 8, under the influence of the flowing current, cools down and cools down the bearing layer of the underwear. Heat transferred from male testicles is transported through the connectors LK to the belt PA where it is dispersed. The whole process is controlled by the microprocessor 1. The microprocessor 1 sends the measured values of temperature, humidity, pulse, skin resistance and battery 10 status to the mobile applications. The algorithm implemented in the processor 1 regulates the amperage flowing through the Peltier cells 8 which directly affects the temperature of the cells 8. The processor 1, using a radio module, communicates over the radio module 2 with the mobile application 12. The mobile application 12 allows for the monitoring the data from the sensors 3, 4, 5, 6, in particular temperature and humidity of the skin, saturation and pulse and skin resistance, also allows for setting the temperature of the Peltier cell 8. The interactive male underwear allows for both cooling and heating the skin surface. The interactive male underwear is equipped with battery power supply, optimally li-pol cell with standard microusb port that allows for charging the cell with a charger. In addition, the layer conducting heat can also be copper foil sewn inside the fabrics.

### LIST OF DESIGNATIONS IN THE FIGURES

UC—skin temperature control system
EN—bearing element—material soaked in silver nanporacticles
PA—belt with heat exchanger
LK—connector
OT—hole
OW—ventilation hole
1—processor
2—radio module
3—temperature sensor
4—humidity sensor
5—pulse oximeter
6—GSR sensor (resistance of the skin)
7—key engaging the cell (PWM)
8—Peltier cell
9—power supply system—Li-Ion cell controller
10—power supply
11—charger
12—mobile application

1. The interactive male underwear comprising the cooling system, characterized in that the bearing element (EN) is connected with the belt (LK) using connectors (LK), however at least one Peltier cell (8) of the skin temperature control system (UC) is mounted on a bearing element (EN).

2. Underwear according to claim 1, characterized in that the skin temperature control system (UC) is mounted on the bearing element (EN).

3. Underwear according to claim 1, characterized in that the skin temperature control system (UC) is mounted on the belt (PA), and at least one Peltier cell (8) of the skin temperature control system (UC) is mounted on bearing element (EN).

4. Underwear according to claim 1, characterized in that the bearing element (EN) is made of woven belt narrowed at the bottom in the capital “U” letter shape, however the upper part of the bearing element (EN) is connected directly with the belt (PA), and the lower part is connected with the belt (PA) using connectors (LK).

5. Underwear according to claim 2, characterized in that the middle part of the bearing element (EN) has a hole (OT).

6. Underwear according to claim 1, characterized in that the bearing element (EN) is made of plastic narrowed at the bottom and with a recess from the top in the shape of capital U letter, however the upper and lower part of the bearing element (EN) is connected with the belt (PA) using the connectors (LK), moreover a ventilation hole (OW) is made in the bearing element (EN).

7. Underwear according to claim 1, characterized in that the bearing element (EN) is equipped with a pocket, where the skin temperature control system (UC) is placed.

8. Underwear according to claim 1, characterized in that the bearing element (EN) is made of heat conducting material.

9. Underwear according to claim 1, characterized in that the belt (PA) is made of heat conducting material.

10. Underwear according to claim 1, characterized in that the belt (PA) is equipped with a heat exchanger.

11. Underwear according to claim 10, characterized in that the heat exchanger has the heat sink and fan.

12. Underwear according to claim 1, characterized in that the internal layer of the belt (PA) is made of a thermal insulating material.

13. Underwear according to claim 10, characterized in that the heat exchanger is a wide belt (PA).

14. Underwear according to claim 1, characterized in that the connector (LK) is made of heat conducting material.

15. Underwear according to claim 1, characterized in that the belt (PA), connector (LK) and bearing element (EN) are made of fabric, preferably of cotton fabric, soaked in silver nanoparticles.

16. Underwear according to claim 1, characterized in that the skin temperature control system (UC) is fitted with a processor (1) connected to the radio module (2), temperature sensor (3), moisture sensor (4), pulse oximeter (5), GSR sensor (6), at least one Peltier cell (8) using a key engaging the cell (7) and power supply (10) via the power control system (9).

17. Underwear according to claim 16, characterized in that the power supply (10) is a battery.

18. Underwear according to claim 16, characterized in that the power supply (10) is an accumulator connected to the charger (11).

19. Underwear according to claim 16, characterized in that radio module (2) is connected wirelessly with the mobile application (12).
